(19) United States
(12) Patent Application Publication
(10) Pub. No.: US 2025/0019635 A1
(43) Pub. Date: Jan. 16, 2025

(54) A BIOREACTOR COMPRISING A BAFFLE

(71) Applicant: Oribiotech LTD, Greater London, London (GB)

(72) Inventors: Farlan Veraitch, Greater London, London (GB); William Raimes, Greater London, London (GB); Shaun Mansfield, Greater London, London (GB); Martin Hoole, Hertfordshire (GB); Nicholas Harding, Hertfordshire (GB); Jason Palmer, Hertfordshire (GB)

(21) Appl. No.: 18/713,525

(22) PCT Filed: Nov. 10, 2022
(86) PCT No.: PCT/GB2022/052856

§ 371 Date: May 24, 2024

(30) Foreign Application Priority Data
Nov. 25, 2021 (GB) 2117018.8

(51) Int. Cl. C12M 1/00; C12M 1/12; C12M 3/00

(52) U.S. Cl. CPC C12M 27/20; C12M 23/26; C12M 23/42; C12M 29/06; C12M 37/00

(57) ABSTRACT

A bioreactor for cell culturing comprising a container having a base portion and a side wall defining an internal volume. The bioreactor also has a lid that closes the internal volume of the container. The base is moveable toward and away from the lid. The bioreactor also includes a baffle member mounted to the lid and positioned in the internal volume of the container such that the baffle member is arranged to mix contents of the bioreactor as the base is moved relative to the lid.

### CROSS-REFERENCE TO RELATED APPLICATIONS

This application is a national phase entry under 35 U.S.C. § 371 of International Patent Application PCT/GB2022/052856, filed Nov. 10, 2022, designating the United States of America and published as International Patent Publication WO 2023/094794 A1 on Jun. 1, 2023, which claims the benefit under Article 8 of the Patent Cooperation Treaty to British Patent Application Serial No. GB 2117018.8, filed Nov. 25, 2021.

### TECHNICAL FIELD

This disclosure relates to a bioreactor comprising a baffle. The bioreactor may be for cell culturing, and the baffle may be provided to mix fluid contents of the bioreactor.

### BACKGROUND

Cell and gene therapy manufacturing processes are often complex and include manual or semi-automated steps across several devices. Equipment systems used in various steps, or unit operations, of cell-based therapeutic products (CTP) manufacturing may include devices for various functions. These various functions may be, for example, cell collection, cell isolation, cell selection, cell expansion, cell washing, volume reduction, cell storage or transportation. The unit operations can vary immensely based on the manufacturing model (i.e., autologous versus allogenic), cell type, intended purpose, among other factors. In addition, cells are “living” entities sensitive to even the simplest manipulations, for example, such as differences in a cell transferring procedure. The role of cell manufacturing equipment in ensuring scalability and reproducibility is an important factor for cell and gene therapy manufacturing.

In addition, cell-based therapeutic products (CTP) have gained significant momentum thus there is a need for improved cell manufacturing equipment for various cell manufacturing procedures. These manufacturing procedures, may include, for example, stem cell enrichment, generation of chimeric antigen receptor (CAR) T cells, and various cell manufacturing processes such as collection, purification, gene modification, incubation, recovery, washing, infusion into a patient, or freezing

The culture or processing of cells typically requires the use of a device to hold the cells, for example, in an appropriate culture medium when culturing the cells. The known devices include shaker flasks, roller bottles, T-flasks, bags and the like. Such devices are typically required to be connected to other devices, such as containers, interfaces or the like, so that various media may be introduced to, or removed from, the device holding the cells. Typically, cells in a culture medium can be added to the device from a flexible bag that is attached using a connecting tube. Alternatively, cells can be transferred by a pipette or by a syringe.

During cell culturing or processing the contents of a bioreactor may be mixed or agitated. It is known to achieve this by moving the bioreactor (e.g., tilting, rotating), or by using a vibratory mixer to agitate the contents of a bioreactor.

### BRIEF SUMMARY

In accordance with the present disclosure there is provided a bioreactor for cell culturing, the bioreactor comprising:
a container having a base and a side wall defining an internal volume, and a lid closing the internal volume, wherein the base is moveable toward and away from the lid; and
a baffle member mounted to the lid and positioned in the internal volume of the container such that the baffle member is arranged to mix contents of the bioreactor as the base is moved relative to the lid.

As the base is moved relative to the lid a fluid level in the container changes and creates relative movement of the fluid and the baffle such that the baffle causes mixing and agitation of the fluid contents of the container. Such mixing and agitation may re-suspend settled or sedimented cells or circulate media or dissolved gases through the fluid.

In the examples, the baffle member may comprise a substantially planar member extending across the internal volume of the container. In particular, the baffle member may extend substantially parallel to the lid. In the examples, the baffle member may comprise a substantially planar surface facing the base of the container.

Accordingly, during movement of the base, fluid is forced around the outside of the baffle member and thereby mixed and agitated. In addition, the baffle member may create pressure differentials in the fluid that cause mixing of fluid that does not pass around the edge of the baffle member.

In the examples, the baffle member may be sized to extend across at least 50 percent of the cross-sectional area of the container, for example, at least 75 percent of the cross-sectional area of the container.

In some examples, the substantially planar surface of the baffle member is non-parallel to the lid. That is, the substantially planar surface of the baffle member may be angled with respect to the lid. The substantially planar surface of the baffle member may additionally or alternatively be angled with respect to the base.

In the examples, the baffle member may comprise a sloped surface, for example, a conical surface, arranged to face the lid. Such a sloped surface may help fluid to flow back into the container as the baffle member is withdrawn from the fluid during use.

In the examples, the baffle member may comprise one or more holes or openings for passage of fluid through the baffle member as the base is moved relative to the lid. The one or more holes or openings may be circular or may be arcuate sectors or other shape. The one or more holes or openings may be perforations in the baffle member. The baffle member may include a plurality of holes or openings, and the plurality of holes or openings may have uniform size and/or shape, or varying size and/or shape.

In the examples, one or more of the holes or openings may comprise a helical or angled surface. For example, one or more of the holes or openings may comprise a passage that is angled with respect to the direction of movement of the base relative to the baffle member such that as fluid passes through the one or more holes or openings a vortex or the like is created, increasing mixing and agitation. Similarly, in the examples, one or more of the holes may have a flap arranged to cover the hole as the baffle moves in one direction relative to the fluid, and to pivot open as the baffle moves in the other direction relative to the fluid. Such a flap may increase mixing and agitation of the fluid.

In the examples, the baffle member may comprise one or more ribs. The one or more ribs may be provided on a surface facing the base, and/or on a surface facing the lid, and may act to increase mixing and agitation of the fluid during use.

In the examples, the container is substantially cylindrical. In the examples, an outer edge of the baffle member is substantially circular. In other examples, the outer edge of the baffle member is substantially non-circular, for example, it may have a wavy profile, or it may be square or triangular.

In the examples, the side wall of the container is compressible or collapsible, for example, a bellows wall. Accordingly, the side wall compresses and extends as the base is moved relative to the lid. In other examples, the side wall of the container may be flexible or extensible, for example, elastically extensible, so to permit movement of the base relative to the lid.

In the examples, the baffle member may be movably mounted to the lid. For example, the baffle member may be slidably mounted to the lid for reciprocal movement of the baffle member within the container. The baffle member may be slidably mounted to the lid via a mounting portion that is received in a linear bearing of the lid. One or more guides may prevent rotational movement of the baffle member as it slides.

In other examples, the baffle member may be rotatably mounted to the lid. In such examples, the baffle member may be mounted to the lid via a mounting portion received in a bearing or bushing in the lid. The baffle member may be freely rotatable within the container, or the baffle member may be actively rotated, for example, by a user or by an actuator located outside of the container.

In the examples, the bioreactor may further comprise a second baffle member mounted to the lid. The second baffle member may be axially aligned with the baffle member such that the baffle member and the second baffle member are spaced from the lid by different amounts. Accordingly, the baffle member and second baffle member can act to mix at different levels within the fluid in the container. In some examples, the baffle member and the second baffle member are the same, and in other examples, the member acts to mix at different levels within the fluid in the container. In some examples, the baffle member and the second baffle member are different, for example, different sizes and/or shapes, or with different holes or openings. In particular, one or both of the baffle member and the second baffle member may have any of the features described above with reference to the baffle member.

In some examples, the second baffle member is parallel to the baffle member, for example, parallel to and spaced from the baffle member. In these examples, the baffle member and the second baffle member may be parallel to the lid. In other examples, the second baffle member is non-parallel to the baffle member, for example, angled with respect to the baffle member. In such examples, one of the baffle member and the second baffle member may be parallel to the lid and the other of the baffle member and the second baffle member may be non-parallel to the lid. Alternatively, both the baffle member and the second baffle member may be non-parallel to the lid.

In the examples, the baffle may comprise a fluid path for conveying fluid into and/or out of the container. In particular, the fluid path may extend from an opening in the baffle member, through a mounting portion of the baffle that is attached to the lid, and to an opening on the exterior of the container. In the examples, the baffle member may further comprise a filter member arranged to filter fluid passing through the fluid path. The filter member may cover the opening in the baffle member. The filter member may be a membrane filter or a depth filter. In the examples, the filter member may be configured to retain cells in the container as fluid is extracted from the container through the fluid path.

The fluid path can be used to add fluid to the container by conveying the fluid through the fluid path from the exterior of the container. Additionally or alternatively, the fluid path may be used to extract fluid from the container by submerging the opening in the baffle member (e.g., by submerging the baffle member) and then applying negative pressure to draw fluid through the fluid path.

In the examples, the lid may comprise one or more ports in fluid communication with the opening in the mounting portion of the baffle. The lid may additionally comprise one or more seals, for example, septum seals, for sealing the one or more ports. The one or more ports may include a connector interface for connecting a consumable to the port for adding fluid to, or removing fluid from, the container.

In various examples, the baffle member is attached to the lid via a mounting portion. The mounting portion may be integrally molded with the baffle member or may be attached to the baffle member. The mounting portion may comprise a generally cylindrical extension and the baffle member may be attached to one end of the cylindrical extension, the other end being attachable to the lid. The mounting portion may be screwed or fastened to the lid, for example, the lid may comprise a threaded spigot and the mounting portion may comprise a threaded hole for attachment of the threaded spigot.

According to the present disclosure, there is also provided a bioreactor system comprising a bioreactor as described above, and an agitator arranged to move the base of the bioreactor such that during use the baffle member mixes contents of the bioreactor.

In the examples, the agitator comprises one or more actuators arranged to move the base. The agitator may comprise an agitator plate that engages the base, and may attach to the base, and the one or more actuators may move the agitator plate.

In the examples, in use, the agitator may be configured to position the base such that the baffle member is submerged in a fluid in the container, and reciprocate the base to generate relative movement of the baffle member and the fluid.

Such reciprocal movement of the base generates relative movement of the baffle member and the fluid, causing mixing and agitation within the fluid.

In the examples, the agitator may be configured to reciprocate the base such that the baffle member remains submerged in the fluid. Such an arrangement may reduce impacting between the baffle member and the surface of the fluid, which may create too much agitation and mixing and stress on cells in the fluid according to the cell culturing process being performed.

In other examples, the agitator may be configured to reciprocate the base such that the baffle member is unsubmerged from the fluid for a part of the reciprocal movement. In such an example, the baffle member may consecutively impact the surface of the fluid, which may provide advantageous mixing, agitation, and/or cell stress according to the cell culturing process being performed.

In the examples, the agitator may be configured to reciprocate the base with a stroke length of up to about 50 millimeters, for example, about 20 millimeters. In the examples, the agitator may be configured to reciprocate the base at a frequency of up to about 80 strokes per minute, for example, between about 10 and about 80 strokes per minute, for example, between about 20 and 60 strokes per minute. In the examples, the agitator may be configured to change an angle of the base relative to the baffle member. For example, the agitator may be configured to tilt the base such that the base is angled with respect to the baffle member.

According to a further aspect of the present disclosure, there is also provided a method of cell culturing, the method comprising providing the bioreactor system described above, providing a cell suspension in the container of the bioreactor, and moving the base of the bioreactor such that the baffle member agitates the cell suspension.

As described above, moving the base of the bioreactor may comprise reciprocating the base relative to the lid. In the examples, the base may be positioned such that the baffle member is submerged in a fluid in the container, and the base may be reciprocated to generate relative movement of the baffle member and the fluid.

In the examples, the base may be reciprocated such that the baffle member remains submerged in the fluid. Such an arrangement may reduce impacting between the baffle member and the surface of the fluid, which may create too much agitation and mixing and stress on cells in the fluid according to the cell culturing process being performed.

In other examples, the base may be reciprocated such that the baffle member is unsubmerged from the fluid for a part of the reciprocal movement. In such an example, the baffle member may consecutively impact the surface of the fluid, which may provide advantageous mixing, agitation, and/or cell stress according to the cell culturing process being performed.

In the examples, the base may be reciprocated with a stroke length of up to about 50 millimeters, for example, about 20 millimeters. In the examples, the base may be reciprocated at a frequency of up to about 80 strokes per minute, for example, between about 10 and about 80 strokes per minute, for example, between about 20 and 60 strokes per minute. In the examples, an angle of the base relative to the baffle member may be varied. For example, the base may be tilted such that the base is angled with respect to the baffle member.

In accordance with a further aspect of the present disclosure there is also provided a baffle for a bioreactor having a container and lid, the baffle comprising a baffle member and a mounting portion attachable to the lid of the bioreactor such that the baffle member is positionable within the container.

In such examples, the base of the bioreactor may be moveable with respect to the lid, as per the examples described above, or the container may be rigid such that the base is fixed relative to the lid. In such examples, the baffle, in particular, the baffle member, may be movable relative to the lid (e.g., slidable and/or rotatable) to mix and agitate the fluid in the container.

In various examples, the baffle is attachable to the lid via the mounting portion. The mounting portion may be integrally molded with the baffle member, or may be attached to the baffle member. The mounting portion may comprise a generally cylindrical extension and the baffle member may be attached to one end of the cylindrical extension, the other end being attachable to the lid. The mounting portion may be screwed or fastened to the lid, for example, the lid may comprise a threaded spigot and the mounting portion may comprise a threaded hole for attachment to the threaded spigot.

In particular, the baffle member may be movably mountable to the lid. For example, the baffle member may be slidably mountable to the lid for reciprocal movement of the baffle member within the container. The baffle member may be slidably mountable to the lid via the mounting portion that is received in a linear bearing of the lid. One or more guides may prevent rotational movement of the baffle member as it slides.

In other examples, the baffle member may be rotatably mountable to the lid. In such examples, the baffle member may be mountable to the lid via the mounting portion received in a bearing or bushing in the lid. The baffle member may be freely rotatable within the container, or the baffle member may be actively rotated, for example, by a user or by an actuator located outside of the container.

In the examples, the baffle member may comprise a substantially planar member arranged to extend across the internal volume of the container. In particular, the baffle member may extend substantially parallel to the lid. In the examples, the baffle member may comprise a substantially planar surface arranged to face the base of the container.

Accordingly, during movement of the baffle member relative to the base, fluid is forced around the outside of the baffle member and thereby mixed and agitated. In addition, the baffle member may create pressure differentials in the fluid that cause mixing of fluid that does not pass around the edge of the baffle member.

In the examples, the baffle member may be sized to extend across at least 5 0percent of the cross-sectional area of the container, for example, at least 75 percent of the cross-sectional area of the container.

In some examples, the substantially planar surface of the baffle member is non-parallel to the lid. That is, the substantially planar surface of the baffle member may be angled with respect to the lid. The substantially planar surface of the baffle member may additionally or alternatively be angled with respect to the base.

In the examples, the baffle member may comprise a sloped surface, for example, a conical surface, arranged to face the lid. Such a sloped surface may help fluid to flow back into the container as the baffle member is withdrawn from the fluid during use.

In the examples, the baffle member may comprise one or more holes or openings for passage of fluid through the baffle member as the base is moved relative to the lid. The one or more holes or openings may be circular or may be arcuate sectors or other shape. The one or more holes or openings may be perforations in the baffle member. The baffle member may include a plurality of holes or openings, and the plurality of holes or openings may have uniform size and/or shape, or varying size and/or shape.

In the examples, one or more of the holes or openings may comprise a helical or angled surface. For example, one or more of the holes or openings may comprise a passage that is angled with respect to the direction of movement of the base relative to the baffle member such that as fluid passes through the one or more holes or openings a vortex or the like is created, increasing mixing and agitation. Similarly, in the examples, one or more of the holes may have a flap arranged to cover the hole as the baffle moves in one direction relative to the fluid, and to pivot open as the baffle moves in the other direction relative to the fluid. Such a flap may increase mixing and agitation of the fluid.

In the examples, the baffle member may comprise one or more ribs. The one or more ribs may be provided on a surface facing the base, and/or on a surface facing the lid, and may act to increase mixing and agitation of the fluid during use.

In the examples, the container is substantially cylindrical. In the examples, an outer edge of the baffle member is substantially circular. In other examples, the outer edge of the baffle member is substantially non-circular, for example, it may have a wavy profile, or it may be square or triangular.

In the examples, the baffle may further comprise a second baffle member mounted to the lid. The second baffle member may be attached to the mounting portion. The second baffle member may be axially aligned with the baffle member such that the baffle member and the second baffle member are spaced from the lid by different amounts. Accordingly, during use the baffle member and second baffle member act to mix at different levels within the fluid in the container. In some examples, the baffle member and the second baffle member are the same, and in other examples, the member acts to mix at different levels within the fluid in the container. In some examples, the baffle member and the second baffle member are different, for example, different sizes and/or shapes, or with different holes or openings. In particular, one or both of the baffle member and the second baffle member may have any of the features described above with reference to the baffle member.

In some examples, the second baffle member is parallel to the baffle member, for example, parallel to and spaced from the baffle member. In these examples, the baffle member and the second baffle member may be parallel to the lid. In other examples, the second baffle member is non-parallel to the baffle member, for example, angled with respect to the baffle member. In such examples, one of the baffle member and the second baffle member may be parallel to the lid and the other of the baffle member and the second baffle member may be non-parallel to the lid. Alternatively, both of the baffle member and second baffle member may be non-parallel to the lid.

In the examples, the baffle may comprise a fluid path for conveying fluid into and/or out of the container. In particular, the fluid path may extend from an opening in the baffle member, through a mounting portion of the baffle that is attached to the lid, and to an opening on the exterior of the container. In the examples, the baffle member may further comprise a filter member arranged to filter fluid passing through the fluid path. The filter member may cover the opening in the baffle member. The filter member may be a membrane filter or a depth filter. In examples, the filter member may be configured to retain cells in the container as fluid is extracted from the container through the fluid path.

The fluid path can be used to add fluid to the container by conveying the fluid through the fluid path from the exterior of the container. Additionally or alternatively, the fluid path may be used to extract fluid from the container by submerging the opening in the baffle member (e.g., by submerging the baffle member) and then applying negative pressure to draw fluid through the fluid path.

In the examples, the lid may comprise one or more ports in fluid communication with the opening in the mounting portion of the baffle. The lid may additionally comprise one or more seals, for example, septum seals, for sealing the one or more ports. The one or more ports may include a connector interface for connecting a consumable to the port for adding fluid to, or removing fluid from, the container.

In accordance with a further aspect of the present disclosure there is also provided a bioreactor comprising the baffle described above, and a bioreactor system comprising the bioreactor.

### BRIEF DESCRIPTION OF THE DRAWINGS

Embodiments of the disclosure are further described hereinafter with reference to the accompanying drawings, in which:

FIG. 1 shows a cell processing system that includes a bioreactor;

FIG. 2 schematically illustrates a cell culturing process;

FIG. 3 illustrates the bioreactor;

FIG. 4 illustrates a cross-section of the bioreactor, showing the baffle;

FIGS. 5A to 5C illustrate an example baffle of the bioreactor of FIG. 4;

FIGS. 6 to 10 illustrate further example baffles of the bioreactor of FIG. 4;

FIGS. 11A and 11B illustrate a method of mixing the contents of the bioreactor using the baffle;

FIG. 12 illustrates a further method of mixing the contents of the bioreactor using the baffle;

FIG. 13 illustrates an example baffle that is angled with respect to a base of the bioreactor;

FIG. 14 illustrates a baffle having two baffle members;

FIG. 15 illustrates a baffle having two baffle members that are angled with respect to a base of the bioreactor;

FIG. 16 illustrates reciprocation of the baffle in the container;

FIG. 17 illustrates reciprocation of two baffle members in the container;

FIG. 18 illustrates an agitator for moving the base of the bioreactor; and

FIG. 19 illustrates a baffle with a fluid path.

### DETAILED DESCRIPTION

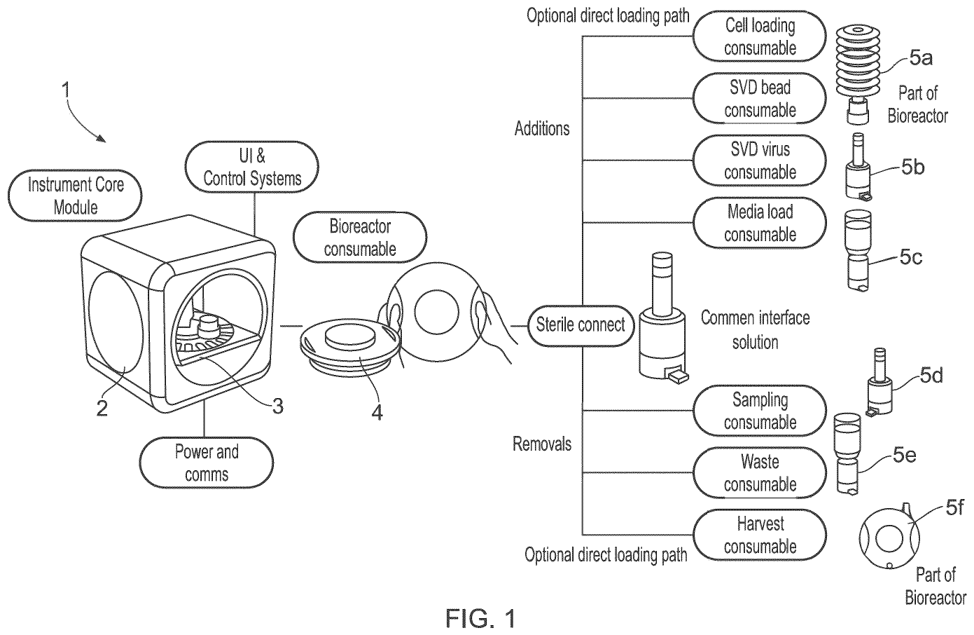

FIG. 1 shows a cell processing system 1 that includes a cell processing housing 2, a cell processing platform 3, a bioreactor 4, and various accessories, for example, “consumables” 5a-5f.

The cell processing housing 2 provides a closed environment for the cell processing platform 3 and is provided with power, connectivity and other utilities needed for the cell processing as described hereinafter. The cell processing platform 3 is adapted to receive the bioreactor 4 and support the bioreactor 4 within the cell processing housing 2. The cell processing platform 3 may include various components and systems that interact with the bioreactor 4 and/or the consumables 5a-5f. For example, the cell processing platform 3 may include an agitator that acts to agitate the bioreactor 4 so as to agitate a cell suspension provided within the bioreactor 4. In other examples, the cell processing platform 3 may include an accessory support arm adapted to hold one or more consumables 5a-5f. In the examples, the cell processing platform 3 may include an actuator operable to actuate one or more of the consumables 5a-5f. The cell processing platform 3 may be configured for automated operation of the cell processing system 1 or may permit manual operation.

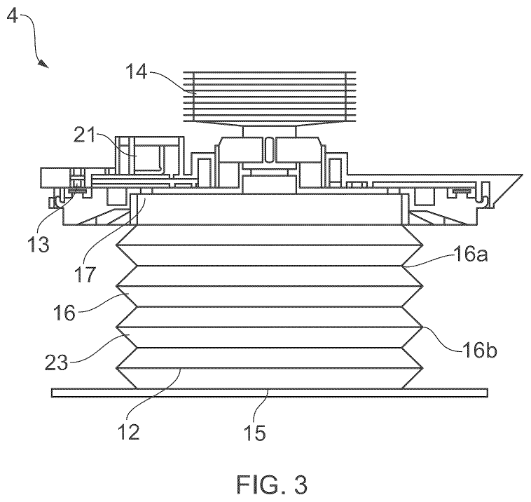

The bioreactor 4, described in more detail with reference to FIG. 3, includes a container 12 and lid, hereinafter called an interface plate 13. During use the container 12 holds a fluid in which the cell processing occurs. In particular, the fluid comprises a population of cells present in a liquid medium. The container 12 may be expandable, for example, by having a bellows wall. The bioreactor 4 is held in the cell processing housing 2 such that the container 12 can expand and retract as it is filled and emptied. The interface plate 13 may be engaged by the cell processing platform 3 and provides various functions relating to the bioreactor 4. For example, the interface plate 13 may have one or more connectors for transfer of fluids into and out of the container 12.

The consumables 5a-5f are for connecting to the bioreactor 4, optionally via the cell processing platform 3, in order to facilitate process steps of the cell culturing process.

In the examples, a cell delivery consumable 5a is provided. The cell delivery consumable 5a is adapted to connect to the bioreactor 4 and deliver a cell suspension to the bioreactor 4. In particular, the cell delivery consumable 5a has a container that is filled with a cell suspension, and a connector that connects to the bioreactor 4 (optionally via the cell processing platform 3). The cell delivery consumable 5a is operable to transfer the cell suspension from the cell delivery consumable 5a into the bioreactor 4. The cell suspension may include “live” cells and a medium. Accordingly, the cell delivery consumable 5a delivers the cell suspension to a bioreactor 4.

The population of cells may comprise any cell type. Suitably the population of cells may comprise a homogenous population of cells. Alternatively, the population of cells may comprise a mixed population of cells.

The population of cells may comprise any human or animal cell type, for example: any type of adult stem cell or primary cell, T cells, CAR-T cells, monocytes, leukocytes, erythrocytes, NK cells, gamma delta t cells, tumor-infiltrating t cells, mesenchymal stem cells, embryonic stem cells, induced pluripotent stem cells, adipose derived stem cells, Chinese hamster ovary cells, NSO mouse myeloma cells, HELA cells, fibroblasts, HEK cells, insect cells, organoids etc. Suitably the population of cells may comprise T-cells.

Alternatively, the population of cells may comprise any microorganism cell type, for example: bacterial, fungal, Archaean, protozoan, algal cells.

In the examples, a fluid delivery consumable 5b is provided. The fluid delivery consumable 5b may hold a particle suspension, for example, a suspension of magnetic particles. The magnetic particles may be magnetic beads. The fluid delivery consumable 5b is operable to deliver the particle suspension to the bioreactor 4.

In the examples, the fluid delivery consumable 5b may alternatively or additionally hold a virus suspension and deliver the virus suspension to the bioreactor 4.

In the examples, a media delivery consumable 5c may be provided. The media delivery consumable 5c may comprise a container that is filled with one or more media, for example, a cell culturing medium, and a connector that connects to the bioreactor 4. The media delivery consumable 5c is operable to move the medium into the bioreactor. In the examples, the media delivery consumable 5c is collapsible, similar to the cell delivery consumable 5a. The medium may be a liquid.

In the examples, the liquid medium may be any sterile liquid capable of maintaining cells. The liquid medium may be selected from: saline or may be a cell culture medium. The liquid medium may be a cell culture medium selected from any suitable medium, for example: DMEM, XVIVO 15, TexMACS. The liquid medium may be appropriate for the type of cells present in the population. For example, the population of cells comprises T cells and the liquid medium comprises XVIVO 10.

In the examples, the liquid medium may further comprise additives, for example: growth factors, nutrients, buffers, minerals, stimulants, stabilizers or the like.

In the examples, the liquid medium comprises growth factors such as cytokines and/or chemokines. The growth factors may be appropriate for the type of cells present in the population and the desired process to be carried out. The liquid medium may comprise stimulants such as antigens or antibodies, which may be mounted on a support. Suitable stimulants are appropriate for the type of cells present in the population and the desired process to be carried out. When culturing T-cells, for example, antibodies are provided as a stimulant in the liquid medium. The antibodies may be mounted on an inert support such as beads, for example: dynabeads.

The additives may be present in the liquid medium at an effective concentration. An effective concentration can be determined by the skilled person on the basis of the population of cells and the desired process to be carried out using known teachings and techniques in the art.

In the examples, the population of cells are seeded in the liquid medium at a concentration of between 1×104 cfu/ml up to 1×108 cfu/ml.

In the examples, a sampling consumable 5d may be provided. The sampling consumable 5d may comprise a sampling vial. In the examples, the sampling consumable 5d may comprise a vacutainer.

In the examples, a waste consumable 5e may be provided. The waste consumable 5e may comprise a container, for example, an expandable container, adapted to receive a waste material removed from the bioreactor 4. The waste consumable 5e may include a filter arranged to filter the cells and/or other media from the fluid within the bioreactor so as only to extract the waste components.

In the examples, a cell harvesting consumable 5f may be provided. The cell harvesting consumable 5f may comprise a container, for example, an expandable container, adapted to receive the cells (and optionally a cell medium) at or toward the end of the cell culturing process. The cell harvesting consumable 5f may include a filter arranged to filter a waste component from the cells and/or other media within the bioreactor so as only to extract the cells and desired media.

In the examples, each of the consumables 5a-5f is connectable to the bioreactor 4 by a common connector. The connector may be that described in applicant's co-pending patent application PCT/GB2020/053229.

The connector can be connected to the consumable 5a-5f, or may be an integral part of the consumable 5a-5f. Operation of the connector, for example, by twisting or sliding, moves a needle so as to create a fluid connection between each end of the connector. Accordingly, the connector allows each consumable 5a-5f to be connected to the bioreactor 4, and then actuation of the connector forms a fluid connection between the consumable 5a-5f and the bioreactor 4 for transfer of materials as set out above. As explained further below, the connectors ensure sterility of the bioreactor 4 and the consumable 5 while creating a fluid connection between the two.

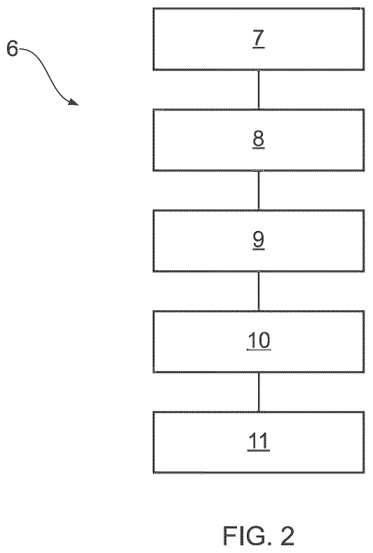

FIG. 2 schematically illustrates a cell culturing process 6 based on the cell processing system 1 described with reference to FIG. 1. As shown in FIG. 2, initially the consumables 5a-5f are prepared 7. For example, a cell delivery consumable 5a may be filled with a cell suspension, and a bead loading consumable 5b may be filled with beads. A connector may be attached to the consumable 5a-5f before or after preparation. Preparation of the consumable(s) 5a-5f may include unpackaging the consumable(s) 5a-5f from a sterile package. It will be appreciated that only the consumables 5a-5f needed for the particular process, and the particular stage of the process, are prepared. For example, some processes would not use beads so a bead loading consumable 5b is not needed, and the cell harvesting consumable 5f is only needed at the end of the cell culturing process 6.

Next, cells are loaded into the bioreactor 4, 8. In particular, a cell delivery consumable 5a is connected to the bioreactor 4 and operated to transfer a cell suspension from the cell delivery consumable 5a into the bioreactor 4. The cell delivery consumable 5a is connected to the bioreactor 4 via a connector, as described above, which forms a fluid connection between the cell delivery consumable 5a and the bioreactor 4.

Either before or after loading cells into the bioreactor 4, 8, the bioreactor 4 is loaded into the cell processing housing 2, 9. In some examples, the bioreactor 4 is attached to the cell processing platform 3 within the cell processing housing 2.

Within the cell processing housing 2, the cells are processed 10 in the bioreactor 4. During cell processing 10 the pressure, temperature, pH and other environmental characteristics within the bioreactor 4 are controlled to ensure that conditions enable cell processing. Cell processing 10 may comprise reprogramming the cells, for example, by using CAR-coding viral DNA. Cell processing 10 may comprise cell culturing.

During cell processing 10 additional consumables 5a-5f may be used to add materials to the bioreactor 4, to extract a sample from the bioreactor 4, and/or to extract waste from the bioreactor 4. For example, a delivery consumable 5b may be used to add magnetic beads to the bioreactor. In the examples, a delivery consumable 5b may be used to add a virus suspension or solution to the bioreactor (e.g., CAR-coding viral DNA). In the examples, a media loading consumable 5c may be used to add one or more media to the bioreactor 4. For example, a media loading consumable 5c may be used to add a balanced salt solution or a basal media to the bioreactor 4. In the examples, a sampling consumable 5d may be used to extract a sample from the bioreactor for testing. In the examples, during or after cell processing 10 a waste consumable 5e may be used to extract a waste media from the bioreactor 4.

After cell processing 10 the cells are harvested 11. Cell harvesting 11 may initially use a waste consumable 5e to extract a waste component. A harvesting consumable 5f can be attached to bioreactor 4 to receive the cells from the bioreactor 4. The cells may be harvested in a media, for example, a cell suspension may be harvested.

As shown in FIG. 3, the bioreactor 4 comprises a container 12 and an interface plate 13. The interface plate 13 comprises at least one connector interface 21 for connecting to an external component, for example, one of the consumables 5a-5f described above. In the examples, the connector interface 21 includes a septum seal that maintains a sealed environment within the container 12 and also permits a needle to pass through to create a fluid connection into the container 12.

The container 12 is a compressible container. In particular, the container 12 has a base portion or bottom wall 15 disposed opposite to the interface plate 13, and a compressible wall 16 defining a sidewall of the container 12. A top part 17 of the compressible wall 16 is attached to the interface plate 13. The top part 17 may include a rigid ring or similar for attaching to the interface plate 13. The compressible wall 16 is compressible such that the bottom wall 15 can move toward and away from the interface plate 13, changing the internal volume of the container 12.

The compressible wall 23 may be a bellows wall, having a concertina arrangement that allows the compressible wall 23 to fold onto itself in order to collapse. In particular, the compressible wall 23 may comprise a series of alternately arranged inward folds 16a and outward folds 16b that allow the compressible wall 23 to collapse like a bellows or concertina. The inward folds 16a and outward folds 16b may be formed by thinned sections in the compressible wall 23, with the inward folds 16a comprise a thinned section arranged on the outer surface of the compressible wall 23, and the outward folds 16b comprising a thinned section arranged on the inner surface of the compressible wall 23.

The container 12 can therefore expand and contract, or be expanded and contracted, according to the material held in the container 12. In particular, the compressible container 12 may expand as the cell culture within the container 12 grows, and/or as additional materials are added. The cell processing housing (2, see FIG. 1) may comprise an actuator adapted to move, for example, push and/or pull, the bottom wall 15 of the container 12 and/or the interface plate 13 to change the volume of the container 12.

As illustrated, the interface plate 13 also includes an expansion container 14, otherwise called a breathing container. The expansion container 14 allows for the container 12 to expand and contract without greatly changing the pressure in the container 12. Alternatively or additionally, the expansion container 14 may be operable, for example, by being mechanically or manually compressed or expanded, to expand or retract the compressible wall 16 of the container 12 and thereby change a volume of the container 12. Alternatively or additionally, the expansion container 14 may be operable, for example, by being mechanically or manually compressed or expanded, to alter the pressure within the container 12.

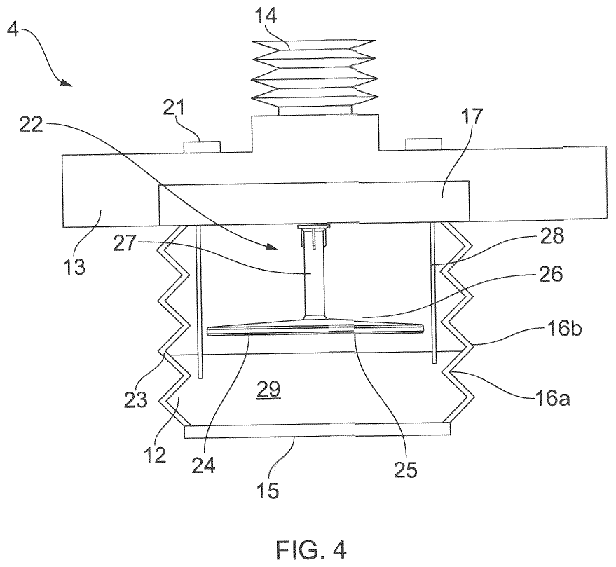

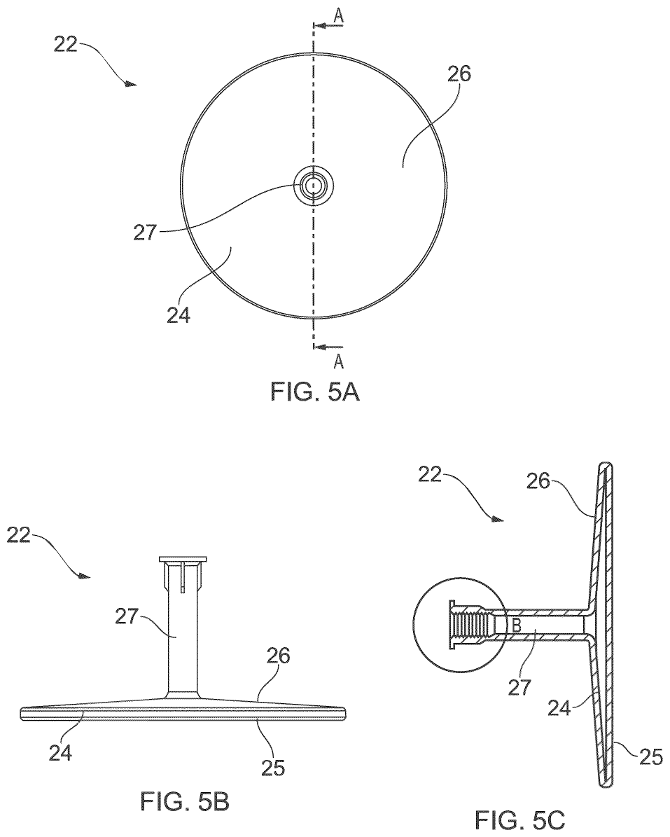

As shown in FIG. 4, which shows a cross-section of the bioreactor 4, the bioreactor 4 includes a baffle 22. The baffle 22 is mounted to the interface plate 13 such that the baffle 22 is suspended within the container 12. As shown in FIG. 4 and also in FIGS. 5A to 5C, the baffle 22 includes a mounting portion 27 that is attachable to the interface plate 13. In the examples, the mounting portion 27 is attachable to the interface plate 13 by a threaded connector, or by a clip or clamp. In the illustrated examples, the mounting portion 27 is attached to the center of the interface plate 13 such that the baffle 22 is centrally positioned within the container 12. However, it will be appreciated that the baffle 22 may be positioned off-center within the container 12. The mounting portion 27 extends from the interface plate 13 toward the base portion 15, and a baffle member 24 is attached to the mounting portion 27 or formed therewith. In this example, the baffle member 24 comprises a substantially flat bottom surface 25 facing the base portion 15 of the container 12. The baffle member 24 also has a conical upper surface 26, facing the interface plate 13. In other examples, the upper surface 26 may be flat (i.e., parallel to the bottom surface 25).

In the examples of FIGS. 4 to 5C, the baffle member 24 is circular and is sized so as to fit within the container 12 of the bioreactor 4. In the examples, the baffle member 24 is sized so as to be spaced from the compressible wall 23 of the container 12. The baffle member 24 may be sized so as to be spaced from the compressible wall 23 of the container 12 by permit a sampling tube 28 to pass between the compressible wall 23 and the baffle member 24 so as to provide a fluid sampling path from the container 12 to the interface plate 13. In the examples, the baffle member 24 may be spaced from the compressible wall 23 by a distance of between about 5 millimeters and about 20 millimeters.

As explained further hereinafter, the baffle 22 is provided to mix contents of the bioreactor 4 during use. In particular, the base portion 15 of the bioreactor 4 may be moved relative to the interface plate 13 and baffle 22 such that the baffle 22 contacts the fluid 29 within the container 12 and mixes it. In the examples, the base portion 15 may be raised and lowered relative to the interface plate 13 (i.e., to change a distance between the base portion 15 and the interface plate 13), and/or the base portion 15 may be tilted relative to the interface plate 13, and/or the base portion 15 may be rotated relative to the interface plate 13.

After mixing the baffle 22 may be moved out of the fluid and the conical upper surface 26 of the baffle member 24 may ensure that fluid is not retained on the baffle 22 and instead runs back into the container 12.

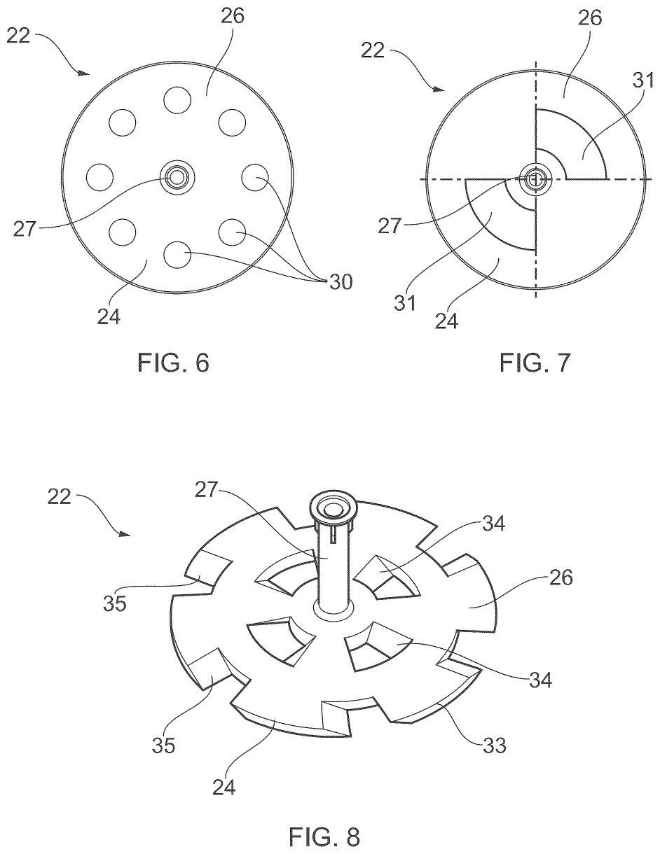

FIGS. 6 to 10 illustrate alternative features of the baffle 22 that can be attached to the interface plate 13 as shown in FIG. 4.

In the example of FIG. 6, the baffle 22, in particular, the baffle member 24, includes one or more holes 30. The holes 30 extend through the baffle member 24, between the lower surface and upper surface 26. The holes 30 may be regularly distributed about the baffle member 24, as shown, or may be irregularly distributed. The holes 30 may have a uniform size or various sizes.

In the example of FIG. 7, the baffle 22, in particular, the baffle member 24, includes one or more openings 31. In the illustrated example, the openings 31 are arcuate sectors of the baffle member 24. The openings 31 extend through the baffle member 24, between the lower surface and upper surface 26. The openings 31 may be regularly distributed about the baffle member 24, as shown, or may be irregularly distributed. The openings 31 may have a uniform size or various sizes. The openings 31 may have the same shape or various shapes. The baffle 22 of FIG. 7 may additionally include one or more holes 30 as shown in FIG. 6.

In the example of FIG. 8, the baffle 22, in particular, the baffle member 24, may comprise one or more angled or helical openings 34. The angled or helical openings 34 extend through the baffle member 24, between the lower surface and upper surface 26. Side walls of the angled or helical openings 34 are angled with respect to the longitudinal direction of the mounting portion 27 and therefore cause increased turbulence in the fluid during use. One or more of the angled or helical openings 35 may extend to the outer edge 33 of the baffle member 24. The angled or helical openings 34 may be regularly distributed about the baffle member 24, as shown, or may be irregularly distributed. The angled or helical openings 34 may have a uniform size or various sizes. The angled or helical openings 34 may have the same shape or various shapes. The baffle 22 of FIG. 8 may additionally include one or more holes 30 as shown in FIG. 6 and/or openings 31 as shown in FIG. 7.

During use, as the baffle 22 is moved through the fluid in the container (12, see FIG. 4), some fluid passes through the holes 30 and/or openings 31 and/or angled or helical openings 34, 35 and other fluid passes around the edge of the baffle member 24. Providing the holes 30 and/or openings 31 and/or angled or helical openings 34, 35 may reduce the volume of fluid being forced around the edge of the baffle member 24 and thereby reduce the velocity of the fluid at the edge of the baffle member 24 and reduce the stresses applied to cells.

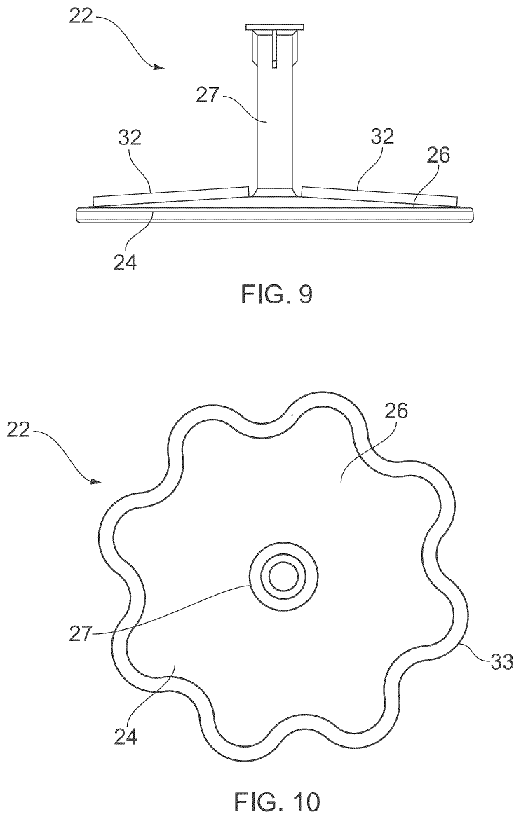

In the example of FIG. 9, the baffle 22 includes one or more ribs 32 formed on the upper surface 26 of the baffle member 24. The ribs 32 may extend radially from the mounting portion 27 toward the edge of the baffle member 24, or they may be angled. The ribs 32 help to mix the fluid in the container (12, see FIG. 4) during use, particularly if the baffle 22 is rotated within the container as described further hereinafter.

In the example of FIG. 10, the outer edge 33 of the baffle member 24 is non-circular. In this example, the outer edge 33 of the baffle member 24 is wavy or undulating. The undulating form of the outer edge 33 of the baffle member 24 provides for uneven flow of fluid over the outer edge 33 of the baffle member 24 during use, which can aid mixing of the fluid.

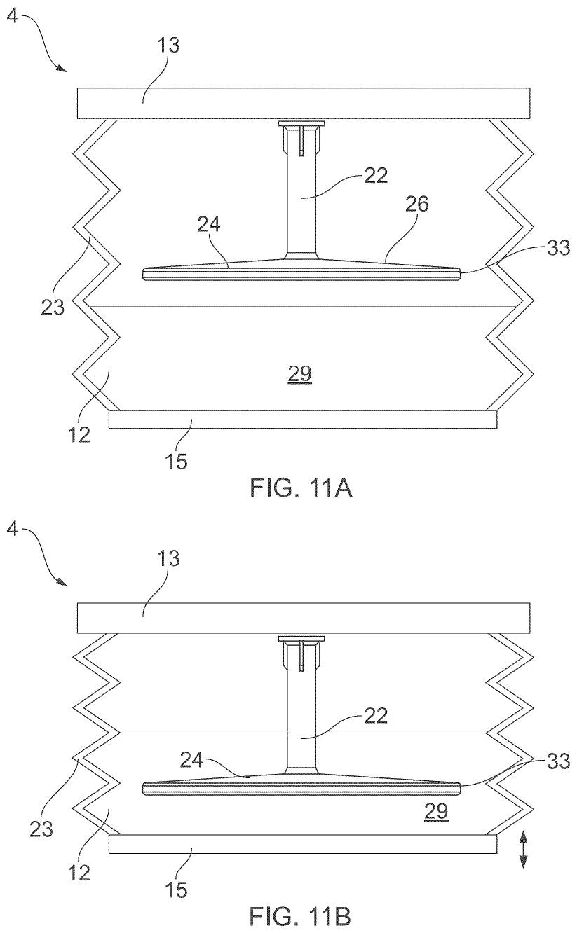

FIGS. 11A and 11B illustrate operation of the bioreactor 4 in a mixing operation. FIG. 11A shows a starting position in which the base portion 15 of the container 12 is spaced from the interface plate 13 such that the baffle 22 is not in contact with the fluid 29 in the container 12. As shown in FIG. 11B, to perform a mixing operation the base portion 15 is raised toward the interface plate 13 until the baffle 22, in particular, the baffle member 24, is submerged within the fluid 29. In the position shown in FIG. 11B, the baffle member 24 is submerged in the fluid 29. From this position, the bottom surface 25 may be reciprocated up and down (toward and away from the interface plate 13) such that the baffle member 24 agitates and mixes the fluid 29.

The base portion 15 may be reciprocated about a starting position corresponding to the baffle member 24 being fully submerged in the fluid 29. The reciprocation preferably has a stroke length such that the baffle member 24 does not contact the base portion 15 and remain submerged in the fluid 29. Keeping the baffle member 24 submerged in the fluid 29 throughout the reciprocal movement prevents the baffle member 24 from impacting on the surface of the fluid 29, which may damage cells. However, in some examples, it may be advantageous to impact the fluid 29 in order to agitate or mix, in which case the base portion 15 may be reciprocated such that the baffle member 24 moves into and out of the fluid 29 and thereby consecutively impacts the surface of the fluid 29 as the base portion 15 is moved up.

In the examples, the starting position of the baffle member 24 in the fluid 29 may be approximately mid-way through the depth of the fluid 29, or it may be closer to the base portion 15 or closer to the top of the fluid 29. Different starting positions may provide different mixing and agitation, particularly when cells have settled or sedimented on the base portion 15.

In the examples, the stroke length of the reciprocation of the base portion 15 may be between about 5 millimeters and about 50 millimeters, for example, about 20 millimeters. In the examples, the stroke rate of the reciprocation of the base portion 15 may be between about 10 strokes per minute and about 80 strokes per minute. In the examples, the stroke speed of the reciprocation of the base portion 15 may be up to about 50 millimeters per second.

As the base portion 15 is reciprocated the relative movement of the baffle member 24 and the fluid 29 causes the fluid to be agitated and mixed. In particular, fluid 29 is forced around the outer edge 33 of the baffle member 24, generating turbulence and flows within the fluid 29 that causes mixing. The turbulence and flows within the fluid 29 may displace settled or sedimented cells to resuspend the cells within the fluid 29, and/or circulate dissolved gases or fluid components (e.g., cell growth media).

After the mixing operation has been completed, the base portion 15 can be lowered back to the position shown in FIG. 11A such that the baffle member 24 is no longer submerged in the fluid 29. The conical upper surface 26 of the baffle member 24 may ensure that fluid is not retained on the baffle member 24.

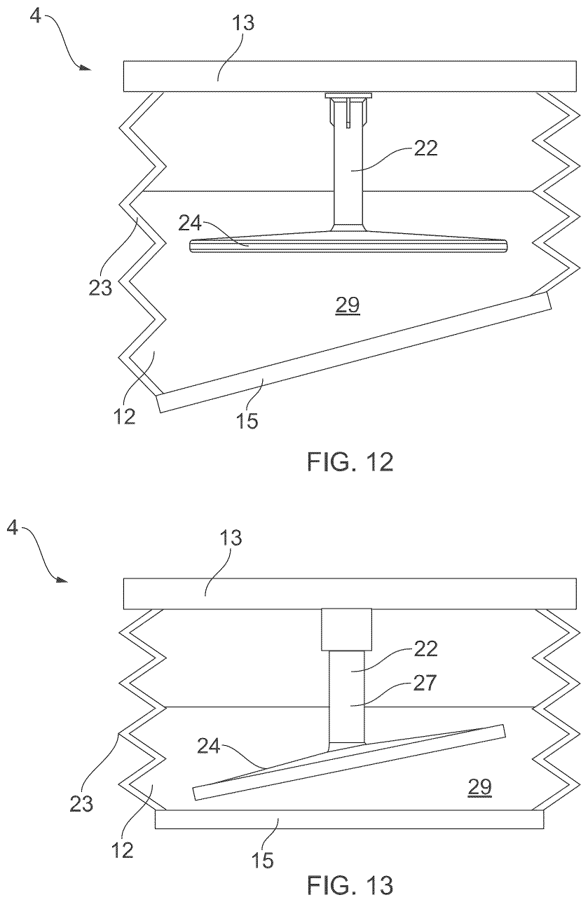

As shown in FIG. 12, during the mixing operation the base portion 15 may be angled with respect to the interface plate 13. In particular, the base portion 15 may be tilted with respect to the interface plate 13 and therefore tilted with respect to the baffle member 24. From the tilted position shown in FIG. 12, the base portion 15 can be raised to submerge the baffle member 24 in the fluid 19, and then reciprocated. The relative angle of the base portion 15 and the baffle member 24 provides different mixing and agitation to as described with reference to FIGS. 11A and 11B.

In some examples, the direction of tilt of the base portion 15 may be varied, for example, cyclically varied, in order to generate relative movement of the baffle member 24 and the fluid 29 to mix and agitate the fluid 29.

In the examples, the tilt angle of the base portion 15 may be varied in order to generate relative movement of the baffle member 24 and the fluid 29 to mix and agitate the fluid 29. In the examples, the tilt angle may be varied by up to about 15 degrees from the horizontal (parallel with the interface plate 13), for example, up to about 10 degrees. The tilt angle may be changed at a rate of up to about 20 degrees per second, which, at a maximum tilt angle of about 10 degrees, is equivalent to about 60 tilts per minute. Such a tilting action may be provided in addition to, or instead of, reciprocation of the base portion 15 toward and away from the interface plate 13.

In the example of FIG. 13, the baffle member 24 may be angled with respect to the interface plate 13 and the base portion 15. In particular, as shown, the baffle member 24 may be tilted relative to the mounting portion 27. The baffle member 24 may be fixed to the mounting portion 27 at a fixed angle, or it may be moveable between a horizontal position as shown in FIG. 12 and the tilted position shown in FIG. 13. In the latter case, the direction and/or angle of the tilt of the baffle member 24 may be varied in order to generate relative movement of the baffle member 24 and the fluid 29 to mix and agitate the fluid 29. In the examples, the tilt angle may be varied by up to about 15 degrees from the horizontal (parallel with the interface plate 13), for example, up to about 10 degrees. The tilt angle may be changed at a rate of up to about 20 degrees per second, which, at a maximum tilt angle of about 10 degrees, is equivalent to about 60 tilts per minute. Such a tilting action may be provided in addition to, or instead of, reciprocation of the base portion 15 toward and away from the interface plate 13. Such a tilting action may be provided in addition to, or instead of, reciprocation of the base portion 15 toward and away from the interface plate 13.

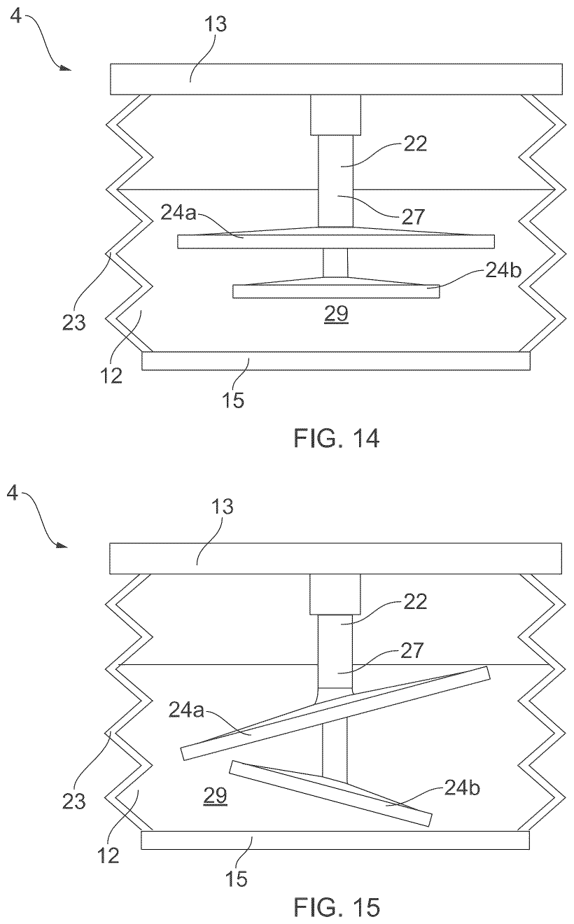

In the example of FIG. 14, the baffle 22 comprises a plurality of baffle members 24a, 24b. In particular, as shown, the baffle 22 may comprise a first baffle member 24a and a second baffle member 24b attached to the mounting portion 27 such that the second baffle member 24b is located between the first baffle member 24a and the base portion 15. Such a baffle 22 may be advantageous for mixing high volumes of liquid as the first and second baffle members 24a, 24b operate at different depths within the fluid 29. As shown, the first and second baffle members 24a, 24b may have different sizes and/or configurations (e.g., holes 30, openings 31, 34, undulating outer edges 33, ribs 32 as described with reference to FIGS. 6 to 10). In the illustrated example, the second baffle member 24b is smaller than the first baffle member 24a.

In the example of FIG. 14, during a mixing operation the base portion 15 may be reciprocated and/or tilted to generate relative movement of the baffle members 24a, 24b and the fluid 29.

As shown in FIG. 15, the first and/or second baffle members 24a, 14b may be tilted with respect to the interface plate 13 and/or with respect to each other in the manner described with reference to FIG. 13. The baffle members 24a, 24b may be tilted in the same direction and by the same angle, or in different directions and/or by different angles.

In the example of FIG. 15, during a mixing operation the base portion 15 may be reciprocated and/or tilted to generate relative movement of the baffle members 24a, 24b and the fluid 29.

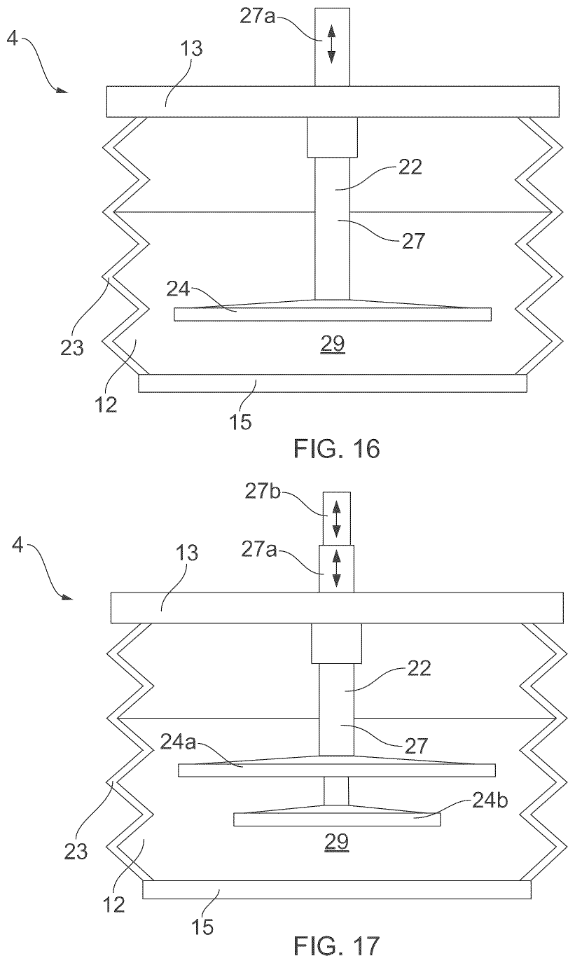

In the example of FIG. 16, the baffle 22 is moveable relative to the interface plate 13. In particular, as illustrated, a first part 27a of the mounting portion 27 may extend through the interface plate 13 and may be actuated (manually or by an actuator) to move the baffle 22, in particular the baffle member 24, relative to the interface plate 13. In this way, the position of the baffle member 24 within the container 12 can be adjusted. The moveable baffle 22 may be provided in addition to the compressible wall element 23, or the moveable baffle 22 may be provided as an alternative to the compressible wall element 23. In particular, in some examples, the container 12 may have rigid or non-compressible sides and the position of the baffle member 24 within the container 12 is controlled by moving the baffle 22 as shown in FIG. 16. In this example, mixing or agitation may be provided by reciprocating the mounting portion first part 27a relative to the interface plate 13 to move the baffle member 24 relative to the fluid 29.

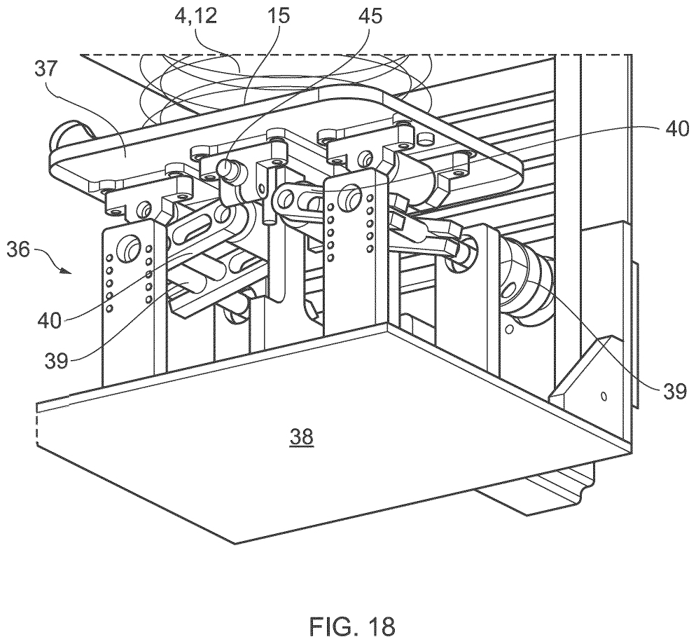

The example of FIG. 17 is similar to the example of FIG. 16, except that the baffle 22 comprises a first baffle member 24a and a second baffle member 24b. The first and second baffle members 24a, 24b may be fixed to each other and moveable relative to the interface plate 13 as described with reference to FIG. 16. Alternatively, as illustrated, the first and second baffle members 24a, 24b may be moveable relative to each other so that a spacing between the first and second baffle members 24a, 24b can be changed. In particular, the mounting member may comprise a first part 27a to which the first baffle member 24a is attached, and a second part 27b to which the second baffle member 24b is attached. The first and second parts 27a, 27b may be independently moveable. The first and second parts 27a, 27b may be telescopically arranged.

In the examples of FIGS. 16 and 17, the or each baffle member, 24, 24a, 24b may additionally or alternatively be rotatable relative to the interface plate 13. In particular, the mounting portion, 27, 27a, 27b may be rotated to rotate the or each baffle member 24, 24a, 24b within the container 12.

In various examples described above, the base portion 15 of the bioreactor 4 moveable relative to the interface plate 13 to mix of agitate the contents. FIG. 18 illustrates an example agitator mechanism 36 that is arranged to move the base portion 15. As shown, the agitator mechanism 36 includes an agitator plate 37 that is moveable to engage the bioreactor 4, in particular the base portion 15. An actuator mechanism is mounted on a base plate 38. Between the base plate 38 and the agitator plate 37 is one or more actuators 39 that act to raise and lower the agitator plate 37 and therefore the base portion 15.

In the illustrated example, the actuators 39 are motors arranged to rotate articulated crank arms 40 that are rotatably connected to the base plate 38 and to the agitator plate 37 such that rotation of the articulated crank arms 40 moves the agitator plate 37. In other examples, linear actuators may be provided to act directly between the base plate 38 and the agitator plate 37.

Supports and guides may guide the movement of the agitator plate 37.

The actuator mechanism may further include a pivotable rod 45 such that the agitator plate 37 can pivot about the pivotable rod 45 to tilt the base portion 15. Pivoting can be provided by raising one linear actuator 39 by a different amount to the other. Accordingly, the agitator plate 37 can be moved relative to the base plate 38 in order to engage the base portion 15 and agitate the contents of the bioreactor 4.

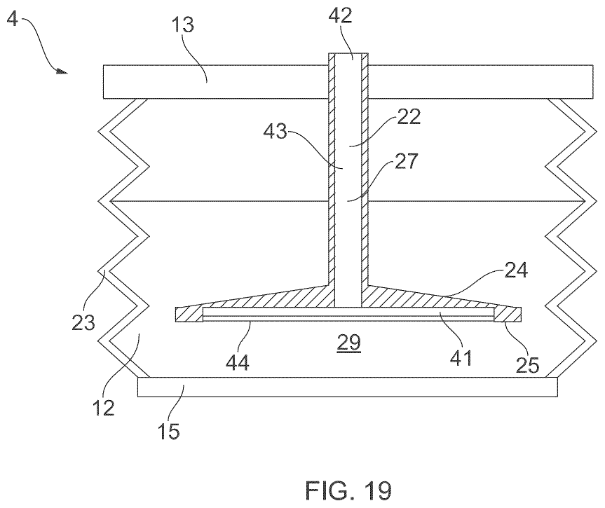

In various examples, the baffle 22 of any of the examples described herein may provide a fluid inlet or fluid outlet in fluid communication between the interior and exterior of the container 12. FIG. 19 shows such an example, with the baffle 22 shown in cross-section. In particular, as shown in FIG. 19, a fluid path 43 is provided between an opening 41 in the bottom surface 25 of the baffle member 24 and an opening 42 in the mounting portion 27, external of the container 12. Accordingly, fluid can be conveyed between the exterior and interior of the container 12 through the fluid path 43. For example, fluid may be added to the container 12 through the fluid path 43, or extracted from the container 12 through the fluid path 43. Fluid can be extracted through the fluid path 43 by at least partially submerging the baffle member 24 and then generating negative pressure at the opening 42 to draw fluid through the fluid path 43. In the examples, a filter member 44, for example, a depth filter or filter membrane, may be provided across the opening 41 in the baffle member 24 to filter the fluid passing into or out of the container 12 through the fluid path 43.

In the examples, agitation of the fluid within the container 12 may be provided by submerging the baffle member 24 and then passing fluid, for example, a media or gas, through the fluid path 43 such that it exits the opening 41 and thereby agitates the fluid in the container 12.

In the examples, the opening 42 may be in communication with one or more ports for forming a sterile connection with an external fluid system, for example, a consumable (5a-5f) as described with reference to FIG. 1. The fluid path 43 provided in the baffle 22 of FIG. 19 may be provided in any of the example baffles 22 described herein. In the examples where the baffle 22 comprises more than one baffle member 24 (e.g., as shown in FIGS. 14, 15, 17), only one or both of the baffle members 24a, 24b may be provided with the opening 41 and optional filter member 44. Preferably, the baffle member 24 closer to the base portion 15 is provided with the opening 41 and optional filter member 44.

Throughout the description and claims of this specification, the words “comprise” and “contain” and variations of them mean “including but not limited to,” and they are not intended to (and do not) exclude other components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

1. A bioreactor for cell culturing, the bioreactor comprising:
a container having a base and a side wall defining an internal volume, and a lid closing the internal volume, wherein the base is moveable toward and away from the lid; and
a baffle member mounted to the lid and positioned in the internal volume of the container such that the baffle member is arranged to mix contents of the bioreactor as the base is moved relative to the lid.

2. The bioreactor of claim 1, wherein the baffle member comprises a substantially planar member extending across the internal volume of the container.

3. The bioreactor of claim 1, wherein the baffle member extends substantially parallel to the lid.

4. The bioreactor of claim 1, wherein the baffle member comprises a substantially planar surface facing the base of the container.

5. The bioreactor of claim 4, wherein the substantially planar surface is non-parallel to the lid.

6. The bioreactor of claim 1, wherein the baffle member comprises a sloped surface arranged to face the lid.

7. The bioreactor of claim 1, wherein the baffle member comprises one or more holes or openings for passage of fluid through the baffle member as the base is moved relative to the lid.

8. The bioreactor of claim 7, wherein one or more of the holes or openings comprises a helical or angled surface.

9. The bioreactor of claim 1, wherein the baffle member comprises one or more ribs.

10. The bioreactor of claim 1, wherein an outer edge of the baffle member is substantially circular.

11. The bioreactor of claim 1, wherein the side wall of the container is compressible.

12. The bioreactor of claim 1, wherein the baffle member is movably mounted to the lid.

13. The bioreactor of claim 12, wherein the baffle member is slidably mounted to the lid for reciprocal movement of the baffle member within the container.

14. The bioreactor of claim 12, wherein the baffle member is rotatably mounted to the lid.

15. The bioreactor of claim 1, further comprising a second baffle member mounted to the lid.

16. The bioreactor of claim 15, wherein the second baffle member is non-parallel to the baffle member.

17. The bioreactor of claim 1, wherein the baffle member comprises a fluid path for conveying fluid into and/or out of the container.

18. The bioreactor of claim 17, wherein the baffle member further comprises a filter member arranged to filter fluid passing through the fluid path.

19. A bioreactor system comprising:
a bioreactor comprising;
a container having a base and a side wall defining an internal volume, and a lid closing the internal volume, wherein the base is moveable toward and away from the lid; and
a baffle member mounted to the lid and positioned in the internal volume of the container such that the baffle member is arranged to mix contents of the bioreactor as the base is moved relative to the lid; and
an agitator arranged to move the base of the bioreactor such that during use the baffle member mixes contents of the bioreactor.

20. The bioreactor system of claim 19, wherein the agitator is configured to:
position the base such that the baffle member is submerged in a fluid in the container, and reciprocate the base to generate relative movement of the baffle member and the fluid.

21. The bioreactor system of claim 20, wherein the agitator is configured to reciprocate the base such that the baffle member remains submerged in the fluid.

22. The bioreactor system of claim 19, wherein the agitator is configured to reciprocate the base with a stroke length of up to about 50 millimetres.

23. The bioreactor system of claim 19, wherein the agitator is configured to reciprocate the base at a frequency of up to about 80 strokes per minute.

24. The bioreactor system of claim 19, wherein the agitator is configured to change an angle of the base relative to the baffle member.

25. A method of cell culturing, the method comprising:
providing a bioreactor system comprising;
a bioreactor comprising:
a container having a base and a side wall defining an internal volume, and a lid closing the internal volume, wherein the base is moveable toward and away from the lid; and
a baffle member mounted to the lid and positioned in the internal volume of the container such that the baffle member is arranged to mix contents of the bioreactor as the base is moved relative to the lid; and
an agitator arranged to move be base of the bioreactor such that during use the baffle member mixes contents of the bioreactor;
providing a cell suspension in the container of the bioreactor; and
moving the base of the bioreactor such that the baffle member agitates the cell suspension.
